(19) United States
(12) Patent Application Publication
(10) Pub. No.: US 2019/0133893 A1
(43) Pub. Date: May 9, 2019

(54) Tooth Paste Gummy

(71) Applicant: Oscar E. Recio Saucedo, Frisco, TX (US)

(72) Inventor: Oscar E. Recio Saucedo, Frisco, TX (US)

(21) Appl. No.: 15/982,069

(22) Filed: May 17, 2018

Related U.S. Application Data
(60) Provisional application No. 62/535,089, filed on Jul. 20, 2017.

(51) Int. Cl. A61K 8/04; A61K 8/02; A61Q 11/00; A61K 8/21; A61K 8/65; A61K 8/26; A61K 8/24; A61K 8/25; A61K 8/46; A61K 8/365; B65D 43/16

(52) U.S. Cl. CPC A61K 8/046; A61K 2800/28; A61Q 11/00; A61K 8/21; A61K 8/0241; A61K 8/65; A61K 8/26; A61K 8/24; A61K 8/25; A61K 8/463; A61K 8/365; B65D 43/169; A61K 2800/87; A61K 2800/72; A61K 8/0204

(57) ABSTRACT

A dental cleaning tablet to provide foaming and cleaning action for oral hygiene is disclosed. The dental cleaning tablet is a convenient gummy that fizz into action when placed in a user's mouth and this comprises special ingredients to create a deep cleaning foam that when used with any toothbrush creates an extraordinary cleaning environment for everyone's oral hygiene regimen. This eliminates the need for a toothpaste and allows for refreshing and complete cleaning solution for teeth, mouth, and gums. The dental cleaning tablets are provided in a compact and convenient casing for extended use.

### BACKGROUND OF THE INVENTION

### A. Technical Field

The present invention generally relates to a dental cleaning tablet. The invention is specifically related to a dental cleaning tablet used to provide foaming and cleaning action for oral hygiene eliminating the need for a toothpaste.

### B. Description of Related Art

Oral hygiene is always an important component for maintaining a healthy lifestyle in a modern society. The health of one's teeth and gums and overall health of the mouth are very critical in maintaining a proper standard of health care for any individual. The hygiene of the mouth is closely associated with the general overall health of the human body. Without a healthy mouth, teeth, and gums the body can not properly intake nutrients and fluids which is required to sustain itself. Regardless of age, good oral health is vitally important to any individual. Diseases that affect the mouth can quickly destroy teeth and other areas of the mouth and can result in an overall decline in an individual's general health.

Practicing good oral hygiene will always include cleaning of the teeth. Utilizing modern techniques, brushing the teeth with a good fluoride toothpaste has always been a good practice and should be done at least twice a day. Cleaning between the teeth often involves flossing or the use of another interdental cleaner. Replacing the toothbrush at regular intervals is also a well-recognized way to maintain good dental health. The health of the gums and the general health of the mouth will always benefit from a good regimen of cleaning the teeth, gums, and mouth on a regular basis. This usually takes more than just brushing the teeth although, that is always a good practice. Baking soda and hydrogen peroxide are common ingredients found in many brands of toothpaste. The foaming and active qualities of these ingredients can greatly assist in the regular cleaning of the teeth and mouth. These ingredients are usually combined with fluoride to create a “complete” cleaning product.

Poor quality oral hygiene only affect an individual's health but also play a role in social status. Bad breath could be offensive to other people and could indicate a serious health problem as well. People are always attracted to a winning smile and fresh breath. Even if the individual doesn't have “perfect” teeth, a clean and fresh mouth is always an asset in any social situation. So, an effective oral hygiene is must for better healthy lifestyle. Currently, variety of toothpastes are used in a tube form conveniently, but it is bulky and sometimes, can be quite messy. It is difficult to get the last bit of paste out of the tube and it could be easily punctured by any sharp means. Especially if left without the cap, the paste can find a way to migrate from the tube to the counter top or other undesired areas for toothpaste.

Currently, the user typically wets the brush, squeezes out a dollop of toothpaste from the tube, and then places the brush in the mouth to begin the brushing process. The user needs to manipulate the tube of paste with one hand while the other is grasping the toothbrush, and this causes lot of inconvenience.

Therefore, there is a need for a dental cleaning solution used to provide foaming and cleaning action for oral hygiene by eliminating the need for a toothpaste.

### SUMMARY OF THE INVENTION

The present invention relates to a dental cleaning tablet used to provide foaming and cleaning action for oral hygiene.

In an embodiment, the dental cleaning tablet is a convenient gummy that fizz into action when placed in a user's mouth and this comprises special ingredients to create a deep cleaning foam when used with any toothbrush and leads to an extraordinary cleaning environment for oral hygiene regimen. This eliminates the need for a toothpaste and allows for refreshing and thorough cleaning of teeth, mouth, and gums for efficient oral health.

In another embodiment, the dental cleaning tablets are provided in a compact and convenient casing for extended use. This casing is a dispensing box which keeps each gummy tablet clean, dry and ready for instant use.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

For the purpose of illustrating the invention, exemplary constructions of the invention are shown in the drawings. However, the invention is not limited to the specific drawings or specific methods or structures disclosed herein. The description of a method step or a structure referenced by a numeral in a drawing is applicable to the description of that method step or structure shown by that same numeral in any subsequent drawing herein.

FIG. 1 illustrates a perspective view of a dental cleaning tablet and a casing to store dental cleaning tablets, incorporating the aspects of the present invention.

FIG. 2 illustrates the user consuming the dental cleaning tablet for cleansing the teeth.

FIG. 3 illustrates a user cleansing with the foaming action of the dental cleaning tablet.

FIG. 4A illustrates a side view of the dental cleaning tablet.

FIG. 4B illustrates a front view of the casing containing the dental cleaning tablet.

### DETAILED DESCRIPTION OF EMBODIMENTS

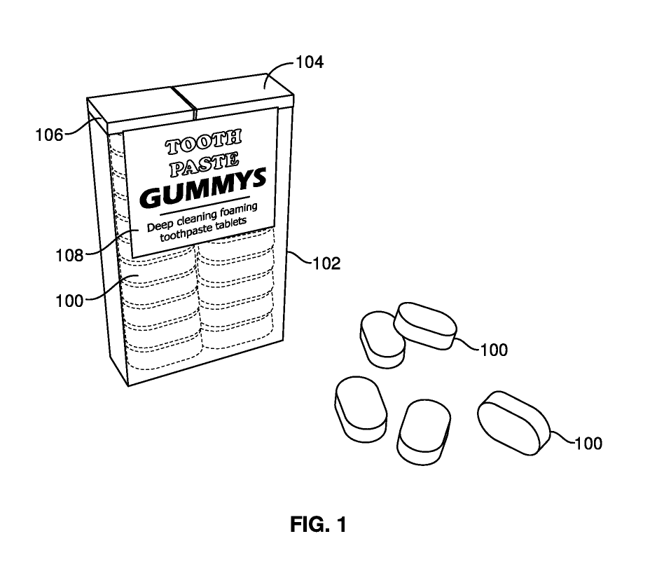

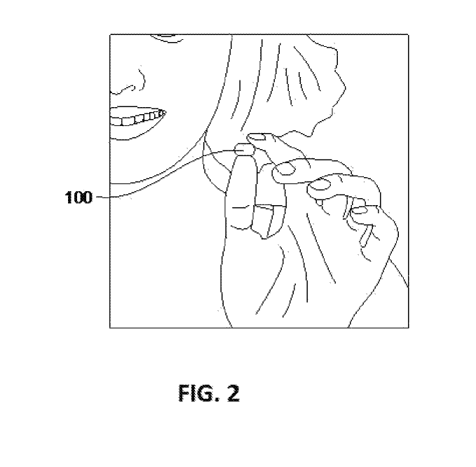

The present invention as shown in FIG. 1 relates to a dental cleaning tablet 100 configured to allow the user to easily brush their teeth and refresh their mouth while brushing their teeth. The dental cleaning tablet 100 provides foaming and cleaning action for oral hygiene without the need for a toothpaste. The dental cleaning tablet 100 is a convenient gummy that fizz into action when placed in a user's mouth and this comprises special ingredients to create a deep cleaning foam when used with any toothbrush 110 and leads to an extraordinary cleaning environment for everyone's oral hygiene regimen as shown in FIG. 2. The user could begin to brush the teeth, driving the foam between the teeth at the gum line, flowing between the teeth to enhance gum cleansing, and foaming throughout the mouth to create the optimum in mouth cleansing. There is no waste because the tablets 100 are perfectly sized. The chemical compounds used in creating the tablets 100 are non-harmful and are typically used in other dental cleansing materials.

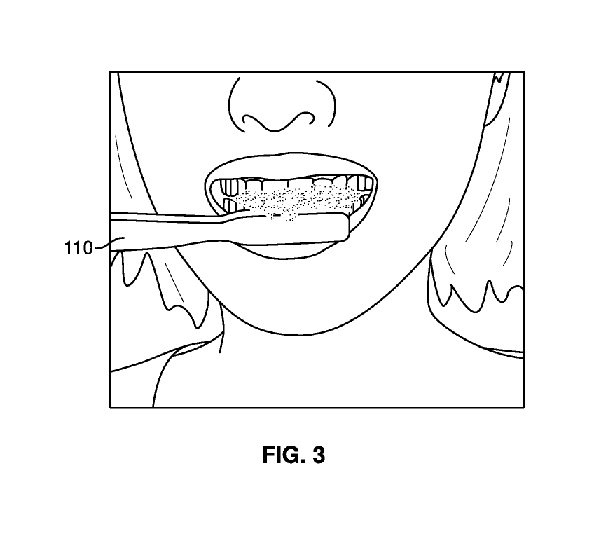

In an embodiment, the composition of the dental cleaning tablet 100 is disclosed. The dental cleaning tablet 100 reacts with the moisture in the mouth to create foaming action using the toothbrush 110 that allows for refreshing and thorough cleaning of teeth, mouth, and gums for efficient oral health as shown in FIG. 3. The dental cleaning tablet 100 is gelatin based and when contacted with saliva allows for foaming action in the mouth. The dental cleaning tablet 100 also includes multi-chemical compounds to provide all the cleansing action of a typical toothpaste while providing a pleasurable and enhanced foaming action in the mouth. This foaming action deep cleans the mouth and allows the dental hygiene chemicals to flow by the teeth roots to enhance plaque removal. Additionally, the foaming action of the dental cleaning tablet 100 also carries fluoride compounds in and around the base of the teeth.

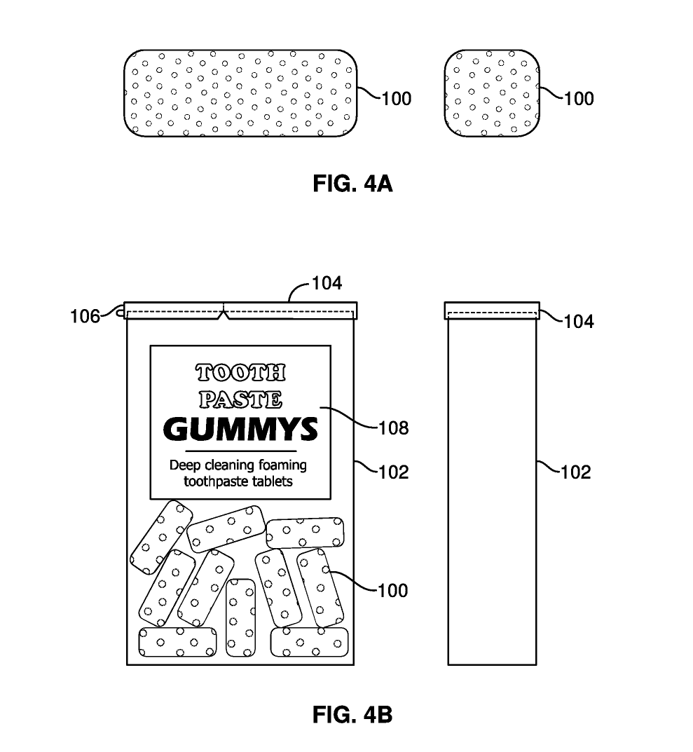

In another embodiment of the present invention as shown in FIG. 4A, manufacturing of the dental cleaning tablet 100 is disclosed. The gelatin compound is the framework upon which the other compounds are retained in the tablet form. The gelatin compound is heated, is incorporated with the other chemicals, dispensed and shaped in corn starch dusted forms. After cooling, the tablets 100 become sugar-free and have a consistency similar to that of gummy bears or many other gelatin based candies. There are several types of abrasives that are commonly used to remove plaque and calculus from the teeth during brushing. These insoluble particles are about 50% of the total volume of product. The most common are aluminum hydroxide (AlOH₃), calcium carbonate (CaCO₃) and hydroxylapatite (Ca₅(PO₄)OH). A small amount of powdered white mica is also used to add a glittering shimmer to the tablet 100. The abrasive effect of the cleansing material should be restricted to a maximum of 50 RDA (relative dentin abrasivity) to prevent excess enamel erosion during use.

In another embodiment as shown in FIG. 4A, the fluorides also make an essential component of the composition of the dental cleaning tablet 100. These chemicals have beneficial effects on the formation of dental enamel and bones. Specifically, Stannous fluoride compound is included in the composition to significantly reduce the incidence of dental caries and to control gingivitis. Typical levels of fluoride in the tablet 100 should be restricted to 1,000 to 1,100 ppm to meet US federal guidelines. The dental cleaning tablet 100 also includes low percentage of sodium lauryl sulfate (SLS) as foaming agents. This detergent compound aids in the foaming of the complex compound when it encounters the saliva in the mouth. The SLS is aided in the foaming action by the inclusion of sodium bicarbonate (NaHCO₃) and citric acid (C₆H₈O₇), which foam and provide a natural antacid as well.

In one embodiment as shown in FIG. 4B, the packaging of the dental cleaning tablets 100 is disclosed. The dental cleaning tablets 100 are provided in a compact and convenient casing for extended use. This casing is a dispensing box 102 which keeps each gummy tablet 100 clean, dry and ready for instant use. The dispensing box 102 is translucent so that the user could see the tablets 100 therein. The dispensing box 102 includes a lid 104 with a living hinge 106 which allows one tablet 100 to get dispensed at a time. The dispensing box 102 is an open top casing joined to a molded top with a living hinged lid 104 that can be opened to dispense the tablet 100. The lid 104 is shaped to snap tightly shut when depressed and can be lifted with a thumb to pop open. In one embodiment, the lid 104 comprises a snap-fit configuration or any other configuration to open or close the lid 104. The lid 104 is adhesively bonded to the top of the casing to make a sealed unit. After emptying the dispensing box 102, it could be recycled to reduce the impact on the environment.

In another embodiment as shown in FIG. 4B, the adhesive preprinted label 108 is provided in the dispensing box 102 to display the product name & foaming action advertising. Federal food labeling is on a pre-printed adhesive label provided on the back of the dispensing box 102. The dispensing box 102 is injection molded with a food grade polypropylene plastic to safely protect the tablets 100 from contamination and allows easy dispensing of individual tablet 100. The dispensing box 102 are machine filled with up to 50 tablets. Once the dispensing box 102 is filled with the tablets 100, it is protected with a shrink wrapped, tamper proof, safety seal, which is removed before use.

In preferred embodiments as shown in FIG. 4B, the dental cleaning tablet 100 comprises flavorants and colorants to provide interest in the use. Four standard flavors such as peppermint, spearmint, cinnamon, and bubblegum are provided. However, other flavors such as peppermint-anise, apricot, lavender, ginger, vanilla, lemon, orange, or other regional favorites could be supplied if the manufacturer marketing department supports the tooling required to produce the flavor. The tablets 100 are colored in various vibrant colors using non-staining natural color compounds. These colorants are food grade, and hypoallergenic materials. The typical colors used are yellow, orange, red, green, or blue. Additionally, the dental cleaning tablet 100 includes remineralizers such as variety of hydroxylaptite (Ca₃(PO₄)₃OH) nanocrystals and calcium phosphates (Ca(H₂PO₄)₂) to accelerate the reformation of tooth enamel. The dental cleaning tablet 100 contains mica particles to improve the sparkling surface of the tablet 100, thereby enhancing the attraction of use by younger children, which improves their dental health.

The preferred embodiments of the present invention disclose the dental cleaning tablet 100 for improving the oral hygiene of a person without using any toothpaste. The tablets 100 are sized to contain the optimum amount of materials to clean the mouth without being wasteful thereby providing a more effective cleansing of the mouth and gums. The pleasurable flavor and the foaming action are activated by the mouth's saliva itself and therefore, the foaming action continues to be enhanced by the generation of additional saliva, until the tablet 100 is totally dissolved. The foaming action and pleasurable flavors leave the mouth feeling exceptionally clean between uses, which are the major advantages of the tablet 100. These gummy tablets 100 replaces the ordinary toothpaste and reduces the mess involved with toothpaste tubes. This is inexpensive and easier way of cleaning the teeth, gums, and entire mouth while freshening the breath. Added flavorants and colorants enhances the interest of use of tablet 100 by users of all age groups.

Although a single embodiment of the invention has been illustrated in the accompanying drawings and described in the above detailed description, it will be understood that the invention is not limited to the embodiment developed herein, but is capable of numerous rearrangements, modifications, substitutions of parts and elements without departing from the spirit and scope of the invention.

The foregoing description comprise illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings presented in the foregoing descriptions. Although specific terms may be employed herein, they are used only in generic and descriptive sense and not for purposes of limitation. Accordingly, the present invention is not limited to the specific embodiments illustrated herein.

What is claimed is:

1. A dental cleaning tablet, comprising:
an amount of toothpaste comprising gelatin,
wherein the tablet is configured to dissolve in saliva in a mouth of a user to directly deliver said toothpaste to said user for utilizing a toothbrush to create foaming and cleaning action.

2. The dental cleaning tablet of claim 1, further comprises fluoride and stannous fluoride.

3. The dental cleaning tablet of claim 2, wherein the fluorides is configured to significantly reduce the incidence of dental caries and to control gingivitis.

4. The dental cleaning tablet of claim 1, further comprises 50 percent of insoluble abrasive particles of the total composition of the tablet.

5. The dental cleaning tablet of claim 1, wherein the insoluble abrasive particles comprises one or more of a group including aluminum hydroxide, calcium carbonate and hydroxylapatite.

6. The dental cleaning tablet of claim 1, provides a relative dentin abrasivity (“RDA”) value of at least 50 RDA to prevent excess enamel erosion.

7. The dental cleaning tablet of claim 1, further comprises a powdered white mica to provide a glittering shimmer to the tablet.

8. The dental cleaning tablet of claim 1, further comprises a foaming agent comprising one or more of a group comprising sodium lauryl sulfate (SLS), sodium bicarbonate (NaHCO3) and citric acid (C6H8O7).

9. The dental cleaning tablet of claim 1, further comprises flavorants and colorants.

10. The dental cleaning tablet of claim 9, wherein the flavorants is selected from one or more of a group comprising peppermint, spearmint, cinnamon, bubblegum, peppermint-anise, apricot, lavender, ginger, vanilla, lemon and orange flavours.

11. The dental cleaning tablet of claim 9, wherein the colorants are made of food grade, and hypoallergenic materials.

12. The dental cleaning tablet of claim 1, further comprises remineralizers including hydroxylaptite (Ca₃(PO₄)₃OH) nanocrystals and calcium phosphates (Ca(H₂PO₄)₂).

13. The dental cleaning tablet of claim 1, is provided in a compact translucent casing, wherein the casing is configured to dispense at least one tablet at a time.

14. The dental cleaning tablet of claim 13, wherein the casing comprises a hinged lid with a snap fit configuration.

15. The dental cleaning tablet of claim 13, wherein the casing is molded with a food grade polypropylene plastic.

16. A method of producing the dental cleaning tablet composition, comprising the step of: heating a gelatin compound and mixing said heated gelatin compound with other chemicals to form a toothpaste mixture; and cooling the mixture to produce a sugar-free dental cleaning tablet composition.
